(19) United States
(12) Patent Application Publication
(10) Pub. No.: US 2023/0377680 A1
(43) Pub. Date: Nov. 23, 2023

(54) METHOD AND SYSTEM FOR FACILITATING PREDICTING THE THREE-DIMENSIONAL STRUCTURE OF AN AMINO ACID SEQUENCE

(71) Applicant: Armand Prieditis, McKinleyville, CA (US)

(72) Inventor: Armand Prieditis, McKinleyville, CA (US)

(21) Appl. No.: 17/747,365

(22) Filed: May 18, 2022

(51) Int. Cl. G16B 15/20; G16B 5/20; G16B 30/00

(52) U.S. Cl. CPC G16B 15/20; G16B 5/20; G16B 30/00

(57) ABSTRACT

One embodiment of the subject matter can facilitate three-dimensional protein structure prediction from an amino acid sequence based on dynamic programming and a probabilistic model learned from training examples. This embodiment is efficient, accurate, can easily be parallelized, and guarantees a prediction that is a most likely three-dimensional protein structure from the amino acid sequence. Moreover, this embodiment is rotation invariant and not require physical or biological knowledge to determine a protein's three-dimensional configuration based on a corresponding amino acid sequence.

### BACKGROUND

### Field

The subject matter relates to predicting the three-dimensional structure of an amino acid sequence. Amino acids are the building blocks of proteins. Currently, there are twenty-one proteinogenic amino acids in humans: alanine, arginine, asparagine, aspartic acid, cysteine, glutamine, glutamic acid, glycine, histidine, isoleucine, leucine, lysine, methionine, phenylalanine, proline, selenocysteine, serine, threonine, tryptophan, tyrosine, and valine. Proteins play numerous critical roles in the body, functioning as enzymes, structural components, antibodies, messengers, and in the transport of atoms and small molecules within cells and throughout the body.

### Related Art

In accepting the 1972 Nobel Prize in Chemistry, Christian Anfinsen famously said that a protein's amino acid sequence should fully determine its three-dimensional structure. This statement has sparked nearly five decades of research aimed at predicting a protein's three-dimensional structure based on its amino acid sequence.

This prediction problem is difficult solve because a typical protein can fold into 10³⁰⁰ possible three-dimensional structures. Even running the fastest current computer since the Big Bang, the enumeration of all possible three-dimensional structures for a typical protein would just be getting started. In contrast, proteins in nature spontaneously fold into their three-dimensional structure in milliseconds. Not only have no computational methods been developed that can predict a three-dimensional structure that quickly, but even our best current prediction methods are relatively inaccurate, especially when no homologous structures exist.

Roughly 200 million proteins (amino acid sequences) are known, but the three-dimensional structure is known for only a tiny fraction of these. The lack of three-dimensional structures for proteins is because the direct method, X-ray analysis, is expensive and difficult to apply to a large number of proteins. An efficient way to predict the secondary structure of proteins based on the known three-dimensional structures of proteins in a repository might be a solution to this bottleneck. In particular the repositories SWISS-MODEL, Genome3D end ModBase provide free access to a large numbers of protein structures. One promising avenue is to machine learn a three-dimensional structure prediction model based on training data comprising amino acid sequences for which a three-dimensional structure is known.

Most recently, AlphaFold became the first machine learning prediction model that could predict a protein's three-dimensional structure with relatively high accuracy, even when no homologous structures exist. AlphaFold combines deep learning neural networks with physical and biological knowledge about protein structure. AlphaFold operates in two stages. First, the trunk of the network processes the inputs through repeated neural network layers. Second, the trunk of the network is followed by the structure module that introduces a three-dimensional structure in the form of a rotation and translation for each residue of the protein.

Although AlphaFold is currently the most accurate three-dimensional structure prediction system, it still has not reached the Christian Anfinsen ideal of determining a protein's three-dimensional structure purely based on the amino acid sequence: it incorporates physical and biological knowledge, both of which are fixed and not learned from data. AlphaFold can also require enormous computational resources and significant hyper-parameter tuning that might not facilitate scaling to larger proteins.

Hence, what is needed is a method and a system for three-dimensional protein structure prediction based on the corresponding amino acid sequence that does not require physical and biological knowledge and that is more efficient and scalable.

### SUMMARY

One embodiment of the subject matter can facilitate three-dimensional protein structure prediction from an amino acid sequence based on dynamic programming and a probabilistic model learned from training examples. This embodiment has several advantages. First, it is more efficient than previous methods to predict three-dimensional protein structure from an amino acid sequence. This efficiency is both in prediction time and learning time. Prediction time is linear in the number of elements in the amino acid sequence. For a non-parallel version, learning time is linear in the average number of elements in the amino acid sequence times the number of training examples. Embodiments of the subject matter can also be parallelized over the training data to yield a learning time that is linear in the maximum number of elements over all amino acid sequences in the training data.

Second, embodiments of the subject matter can fit three-dimensional structures of greater complexity because it is based on a non-linear model and because it propagates local information globally, throughout the sequence. Third, it is optimal in that it guarantees a prediction that is a most likely three-dimensional protein structure from the amino acid sequence for the model. This guarantee is based on optimality of dynamic programming and basic probability.

Fourth, it is general because it is rotation invariant. Fifth, it does not require physical or biological knowledge to determine a protein's three-dimensional configuration based on a corresponding amino acid sequence. Sixth, the framework is simpler to implement than that of Deep Learning methods such as in AlphaFold: no complex hyperparameters to tune, no complex neural network structures to determine, and no specialized hardware required for speed. Seventh, embodiments of the subject matter facilitate greater opportunities for parallelism: across training examples and random restarts for learning and across subclasses for both prediction and learning.

The details of one or more embodiments of the subject matter are set forth in the accompanying drawings and the description below. Other features, aspects, and advantages of the subject matter will become apparent from the description, the drawings, and the claims.

### BRIEF DESCRIPTION OF THE FIGURES

FIG. 1 presents an example system for facilitating graph classification.

In the FIGURES, like reference numerals refer to the same FIGURE elements.

### DETAILED DESCRIPTION

A protein's three-dimensional configuration can be represented in multiple ways. In embodiments of the subject matter, this three-dimensional configuration is represented as a sequence of dihedral angles. More formally, in embodiments of the subject matter, an amino acid sequence comprises an m-length (m≥2) sequence of amino acids.

In embodiments of the subject matter, training data for an amino acid sequence comprises an m−1-length sequence of dihedral angles, each of which is between two contiguous amino acids. That is, there is one fewer dihedral angles than amino acids in the sequence.

Each dihedral angle comprises two angles, one for each plane: α and β. A sequence of such angles uniquely identifies the three-dimensional configuration of the amino acid sequence, regardless of rotation. The two angles between amino acids i and i+1 in the sequence can either use amino acid i for the origin or amino acid i+1 for the origin. However, the choice of origin must remain consistent across the entire sequence and across all training examples. A preferred embodiment of the subject matter assumes amino acid i for the origin and amino acid i+1 as the destination.

In embodiments of the subject matter, the prediction task is to determine the dihedral angles between successive amino acids in a given amino acid sequence. During operation, embodiments of the subject matter can execute the following procedure.

$$\# determine most likely states for each element in the sequence$$

$$s ∈ S : \\ {t}_{1 , s} ← ℒ ( [ \begin{matrix}{x}_{{1}_{γ}} \\ {x}_{{2}_{γ}} \\ o ( s )\end{matrix} ] , {μ}_{γ : τ} {Σ}_{γ : τ , γ : τ} ) \\ {g}_{1 , s} ← s \\ {a}_{1 , s} ← \hat{μ} ( [ \begin{matrix}{x}_{{1}_{γ}} \\ {x}_{{2}_{γ}} \\ o ( s )\end{matrix} ] , θ : θ , γ : τ , μ , Σ ) \\ 2 \leq i \leq m - 1 : \\ s ∈ S : \\ {t}_{i , s} ← {max}_{{s}^{′} ∈ S} \{ l ( [ \begin{matrix}{x}_{{i}_{γ}} \\ {x}_{i + {1}_{γ}} \\ o ( s )\end{matrix} ] \vert [ \begin{matrix}{a}_{i - 1 , {s}^{′}} \\ {x}_{i - {1}_{γ}} \\ o ( {s}^{′} )\end{matrix} ] , γ : τ , {θ}^{′} : {τ}^{′} , \dot{μ} , \dot{Σ} ) + {i}_{t - 1 , s ′} \} \\ {g}_{i , s} ← {argmax}_{{s}^{′} ∈ S} \{ l ( [ \begin{matrix}{x}_{{i}_{γ}} \\ {x}_{i + {1}_{γ}} \\ o ( s )\end{matrix} ] \vert [ \begin{matrix}{a}_{i - 1 , {s}^{′}} \\ {x}_{i - {1}_{γ}} \\ o ( {s}^{′} )\end{matrix} ] , γ : τ , {θ}^{′} : {τ}^{′} , \dot{μ} , \dot{Σ} ) + {t}_{i - 1 , s ′} \} \\ {a}_{i . s} ← \hat{μ} ( [ \begin{matrix}{x}_{{i}_{γ}} \\ {x}_{i + {1}_{γ}} \\ o ( s ) \\ {a}_{i - 1 , {g}_{i , s}} \\ {x}_{i - {1}_{γ}} \\ o ( {g}_{i , s} )\end{matrix} ] , θ : θ , γ : {τ}^{′} , \dot{μ} , \dot{Σ} )$$

First, embodiments of the subject matter determine the most likely states for each element in the sequence. Here, S corresponds to a set of states. Typically, the set of states S={1 . . . k}, where k is a positive integer. States are like mixture components in a mixture model: they are merely identifiers that operate like a subclass in a model. More generally, the set of states can be any finite set of k elements such as {a,b,c,d}. During operation, embodiments of the subject matter treat the set {a,b,c,d} the same as the set {1,2,3,4}. Though the states have different labels, the number of states is the same and hence these two different sets of states will be treated equivalently by embodiments of the subject matter. For convenience of implementation, a preferred embodiment of the subject matter uses states S={1 . . . k}, which is equivalent to any k element set of labels in embodiments of the subject matter.

The expression s∈S: corresponds to a “for” loop that is executed for every state s∈S. For each state, for each element in the sequence, t_{i,s} stores a log maximum likelihood based on position i and state s. Similarly, g_{i,s} stores the predecessor state based on position i and state s. In contrast, a_{i,s} stores the most likely dihedral angles based on position i and state s. Embodiments of the subject matter can then use these stored values of t_{i,s}, g_{i,s}, and a_{i,s} to determine t, g, and a for larger values of i and other states based on dynamic programming, which will be described shortly.

The assignment

$${t}_{1 , s} ← ℒ ( [ \begin{matrix}{x}_{{1}_{γ}} \\ {x}_{{2}_{γ}} \\ o ( s )\end{matrix} ] , {μ}_{γ : τ} , {Σ}_{γ : τ , γ : τ} )$$

sets the first value of t for state s, where the values x₁_{_{γ}} and x₂_{_{γ}} correspond to one-hot versions of the amino acids at the first and second locations in the input x, for which the three-dimensional structure is to be determined, the function o(s) returns the one-hot representation of the state, and [symbol] (x, μ, Σ)=ln|Σ|+(x−μ)^{T}Σ⁻¹(x−μ).

The function [symbol] returns the ln (natural log) of the probability of x in a multivariate Gaussian distribution with mean μ and covariance matrix Σ. (The constants such as π and ½ in [symbol] are removed here because they don't affect the outcome in embodiments of the subject matter.) Also, ln|Σ| is the natural log of the determinant of Σ, M^{T} is the transpose of matrix M, and

Σ⁻¹ is the inverse of a square matrix Σ.

Embodiments of the subject matter can simultaneously leverage dynamic programming by using the state and position as an index to save precomputed results and by using a one-hot version of the state to leverage multivariate Gaussians, both in conditional and unconditional distributions. For example t_{1,s} can be precomputed and stored for reuse through dynamic programming and o(s), the one-hot version of s, can be used in a Gaussian distribution because it comprises continuous values (though it is represented as a vector of continuous values, one of which is always a 1 and the rest zeros).

As mentioned above, in embodiments of the subject matter, each amino acid is represented as a one-hot vector. For example, if there are 21 amino acids represented in alphabetical order, the one-hot vector for alanine (the first amino acid in alphabetical order) can be represented as 21-row column vector with a one in the first position and zeroes elsewhere:

$$[ \begin{matrix}1 \\ 0 \\ ⋮ \\ 0\end{matrix} ] .$$

Since an amino acid is not required for indexing, embodiments of the subject matter do not need two representations (original and one-hot) for amino acids. Only a one-hot is needed for input into a multivariate Gaussian distribution.

A one-hot representation is frequently used in machine learning to handle categorical data. In this representation a k-category variable is converted to a k-length vector, where a 1 in location i of the k-length vector corresponds to the i^{th} categorical variable; the rest of the vector values are 0. For example, if the categories are A, B, and C, then a one-hot representation corresponds to a length three vector where A can be represented as

$$[ \begin{matrix}1 \\ 0 \\ 0\end{matrix} ] ,$$

### B as

$$[ \begin{matrix}0 \\ 1 \\ 0\end{matrix} ] ,$$

### and C as

$$[ \begin{matrix}0 \\ 0 \\ 1\end{matrix} ] .$$

Other permutations of the vector can be used to equivalently represent the same three categorical variables.

The subscripts in a matrix refer to blocks of conformably partitioned matrices indexed by the associated symbol. For example, μ_{θ} refers to that block of the mean vector corresponding to the dihedral angles mean, μ_{γ} refers to that block of the mean vector corresponding to an amino acid at a first location, μ_{γ}_{⁺} refers to that block of the mean vector corresponding to an amino acid at a next location, and μ_{τ} refers to that block of the mean vector corresponding to the state.

The covariance matrix Σ is similarly conformably partitioned as follows. The mean vector μ is conformably partitioned as

$$[ \begin{matrix}{μ}_{θ} \\ {μ}_{γ} \\ {μ}_{{γ}^{+}} \\ {μ}_{τ}\end{matrix} ] ,$$

the covariance matrix Σ is conformably partitioned as

$$[ \begin{matrix}{Σ}_{θ , θ} & ⋯ & {Σ}_{θ , τ} \\ ⋮ & ⋱ & ⋮ \\ {Σ}_{τ , θ} & ⋯ & {Σ}_{τ , τ}\end{matrix} ] ,$$

the second mean vector \dot{μ} is conformably partitioned as

$$[ \begin{matrix}{\dot{μ}}_{θ} \\ {\dot{μ}}_{γ} \\ {\dot{μ}}_{{γ}^{+}} \\ {\dot{μ}}_{τ} \\ {\dot{μ}}_{{θ}^{′}} \\ {\dot{μ}}_{{γ}^{′}} \\ {\dot{μ}}_{{τ}^{′}}\end{matrix} ] ,$$

and the second covariance matrix \dot{Σ} is conformably partitioned as

$$[ \begin{matrix}{\dot{Σ}}_{θ , θ} & ⋯ & {\dot{Σ}}_{θ , {τ}^{′}} \\ ⋮ & ⋱ & ⋮ \\ {\dot{Σ}}_{{τ}^{′} , θ} & ⋯ & {\dot{Σ}}_{{τ}^{′} , {τ}^{′}}\end{matrix} ] .$$

The prime (′) notation refers to an immediate predecessor in the sequence. For example, \dot{μ}_{θ′} refers to that block of the \dot{μ} vector for a dihedral angle before the dihedral angle associated with \dot{μ}_{θ}. The plus (+) superscript notation refers to an immediate successor in the sequence.

The range notation a: b follows the order of variables that appear in μ, Σ, \dot{μ} and \dot{Σ}. For example, γ: τ′ specifies a range of blocks from γ to τ′: γ, γ⁺, τ, θ′, γ′, τ′. This range notation is merely a compact and succinct way to specify blocks of a conformably partitioned vector or matrix. The block order described here facilitates simpler notation of these ranges for the particular uses in embodiments of the subject matter.

An advantage of a multivariate Gaussian distribution in the likelihood function [symbol] is that a missing value can simply be ignored. Only those blocks corresponding to known variables in the mean vector and covariance matrix are required to produce the same result as if marginalizing over missing variables.

The assignment g_{1,s}←s sets the first value of g to s for the state s. The assignment

$${a}_{1 , s} ← \hat{μ} ( [ \begin{matrix}{x}_{{1}_{γ}} \\ {x}_{{2}_{γ}} \\ o ( s )\end{matrix} ] , θ : θ , γ : τ , μ , Σ )$$

sets the first dihedral angles for a at state s to the most likely dihedral angles given x₁_{_{γ}}x₂_{_{γ}}, o(s). The value a_{1,s} is a two-row column vector corresponding to the most likely dihedral angle at the first location for state s (recall that the dihedral value corresponds to two angles, α and β).

Here, \hat{μ}(x, a, b, μ, Σ)=μₐ+Σ_{a,b}Σ_{b,b}⁻¹(x−μ_{b}), which is the conditional mean of a multivariate Gaussian distribution. In the function \hat{μ}, the variable a corresponds to a block associated with the dihedral angles and the variable b corresponds to the block associated with the rest of the variables that are known at the time of dihedral angles prediction for this index and state.

The three initial assignments set the base values for t, g, and a, will be used to set values for t, g, and a later in the sequence through dynamic programming. An alternative to the base values and μ and Σ is to include a dummy amino acid and dihedral angles at a dummy border (first location), and only use \dot{μ} and \dot{Σ}, and a “for” loop, which will be described shortly.

Although such dummy borders are common in image processing to reduce code, the problem with dummy borders is that a dummy state is required for those edges as well as dummy values such as the amino acid and dihedral angles. Zeros are often used as for such values associated with dummy borders, but this can bias the values of \dot{μ} and \dot{Σ}, especially if zeros are actual values in the rest of the sequence.

A disadvantage of using edge cases instead of borders is that, statistically, there are fewer edge cases and interior cases in the training data. For example, with n k-length sequences, there will only be n edge cases but n×k interior cases. In the spirit of greater clarity and potentially improved accuracy, description of embodiments of the subject matter here avoid minor tricks such as dummy borders to reduce the amount of code.

The expression 2≤i≤m−1: corresponds to a “for” loop that loops through values of i from 2 to m−1, inclusive, where m is the number of elements in the amino acid sequence for which the dihedral angles are to be determined. The assignment

$${t}_{i , s} ← {max}_{{s}^{′} ∈ S} \{ l ( [ \begin{matrix}{x}_{{i}_{γ}} \\ {x}_{i + {1}_{γ}} \\ o ( s )\end{matrix} ] \vert [ \begin{matrix}{a}_{i - 1 , {s}^{′}} \\ {x}_{i - {1}_{γ}} \\ o ( {s}^{′} )\end{matrix} ] , γ : τ , {θ}^{′} : {τ}^{′} , \dot{μ} , \dot{Σ} ) + {i}_{t - 1 , s ′} \}$$

sets t_{i,s} to the log likelihood of the most likely value state value s′ based on the right-hand-side of the assignment, part of which has already been determined and stored in t_{i-1,s′}, where l(x|y, a, b, μ, Σ)= [symbol] (x, \hat{μ}(y, a, b, μ, Σ), \hat{Σ}(a, b, Σ)) and \hat{Σ}(a, b, Σ)=Σₐ−Σ_{a,b}Σ_{b,b}⁻¹τ_{b,a}. \hat{Σ}(a, b, Σ) corresponds to the conditional covariance of a multivariate Gaussian. Together, the mean \hat{μ}(a, b, \dot{μ}, \dot{Σ}, y) and covariance matrix \hat{Σ}(a, b, \dot{Σ}) define a multivariate Gaussian distribution.

The assignment

$${g}_{i , s} ← {argmax}_{{s}^{′} ∈ S} \{ l ( [ \begin{matrix}{x}_{{i}_{γ}} \\ {x}_{i + {1}_{γ}} \\ o ( s )\end{matrix} ] \vert [ \begin{matrix}{a}_{i - 1 , {s}^{′}} \\ {x}_{i - {1}_{γ}} \\ o ( {s}^{′} )\end{matrix} ] , γ : τ , {θ}^{′} : {τ}^{′} , \dot{μ} , \dot{Σ} ) + {t}_{i - 1 , s ′} \}$$

records (saves in memory) the most likely predecessor state s′ for the previous assignment for t_{i,s}. This recording will be used in the next assignment to determine the dihedral angles based on this state and other information and a backtrace method, which will be described.

The assignment

$${a}_{i . s} ← \hat{μ} ( [ \begin{matrix}{x}_{{i}_{γ}} \\ {x}_{i + {1}_{γ}} \\ o ( s ) \\ {a}_{i - 1 , {g}_{i , s}} \\ {x}_{i - {1}_{γ}} \\ o ( {g}_{i , s} )\end{matrix} ] , θ : θ , γ : {τ}^{′} , \dot{μ} , \dot{Σ} )$$

sets a_{i,s} to the most likely dihedral angles given

$$[ \begin{matrix}{x}_{{i}_{γ}} \\ {x}_{i + {1}_{γ}} \\ o ( s ) \\ {a}_{i - 1 , {g}_{i , s}} \\ {x}_{i - {1}_{γ}} \\ o ( {g}_{i , s} )\end{matrix} ] .$$

Note that the quantities a_{i-1,g}_{_{i,s}} and g_{i,s} have been previously been determined through dynamic programming.

The term dynamic programming as used by embodiments of the subject matter is means that quantities precomputed earlier in the sequence can be used to later in the sequence. Dynamic programming is efficient because of this re-use of precomputed data. More generally, dynamic programming can be used to solve an optimization problem by dividing it into simpler subproblems where an optimal solution to the overall problem is based on an optimal solution to the simpler subproblems. In embodiments of the subject matter, the optimization problem is maximization and “simpler” corresponds values that have been precomputed earlier in the sequence. For example, all three of t_{i,s}, g_{i,s}, and a_{i,s} have been determined with dynamic programming because they are all based on previously determined values from earlier in the sequence. In probabilistic terms, a_{i,s} is also determined with optimization, though a closed-form, because the function \hat{μ} returns the most likely value, which is the mean of the conditional multivariate Gaussian distribution.

Once the values for t, g, and a have been determined, embodiments of the subject matter can backtrace from high to low values of the sequence to find a most likely sequence of states for the amino acid sequence. The backtrace procedure is shown below.

$$\# backtrace to find most likely state sequence$$

$${r}_{m - 1} ← {argmax}_{s ∈ S} \{ {t}_{m - 1 , s} \} \\ m - 1 \geq i \geq 2 : {r}_{i - 1} ← {g}_{i , {r}_{i}}$$

The backtrace assignments begin with the penultimate index value, m−1, in the sequence. Specifically, the assignment

$${r}_{m - 1} ← {argmax}_{s ∈ S} \{ {t}_{m - 1 , s} \}$$

stores the most likely state for position m−1 in a sequence of m amino acids.

Subsequently, m−1≥i≥2:rᵢ₋₁←g_{i,r}_{ᵢ} sets the values for the remaining positions from m−1 down to 2. Because the “for” loop runs from m−1 down to 2, the assignment is based on the previously set index value rᵢ. This is another use of dynamic programming in embodiments of the subject matter.

Once these most likely states are found for each index in the sequence, embodiments of the subject matter can determine the dihedral angles based on the most likely states and their associated dihedral angles.

#Determine Dihedral Angles

1≥i≥m−1:hᵢ←a_{i,r}_{ᵢ}

The “for” loop 1≥i≥m−1: hᵢ←a_{i,r}_{ᵢ} determines the dihedral angles for each index. In this case, the loop proceeds from low to high because all values of a_{i,r}_{ᵢ} have already been determined. This loop can be executed in parallel. After the loop completes, the dihedral angles for the position i in the sequence is equal to hᵢ. All of the dihedral angles in the sequence uniquely determine the three-dimensional structure of the protein corresponding to the amino acid sequence.

Embodiments of the subject matter can execute the following steps to learn a prediction model based on a multivariate Gaussian distribution. The first step in learning the prediction model in embodiments of the subject matter randomly initializes the states for each sequence (training example), for each dihedral angle in the sequence. Here, n corresponds to the number of training examples and mⱼ corresponds to the number of elements in the sequence for training example j. The range for i≤mⱼ−1 because there are one fewer dihedral angles than amino acids in the sequence.

```
# randomly initialize states
1 ≤ j ≤ n:
  1 ≤ i ≤ mⱼ − 1:
    rᵢ^{j} ← random(S)
```

$$\# update model \\ data ← ∅ \\ \dot{data} ← ∅ \\ 1 \leq j \leq n : \\ data \cdot append ( [ \begin{matrix}{x}_{{1}_{θ}}^{j} \\ {x}_{{1}_{γ}}^{j} \\ {x}_{{2}_{γ}}^{j} \\ o ( {r}_{1}^{j} )\end{matrix} ] ) \\ 2 \leq i \leq {m}_{j} - 1 : \\ \dot{data} \cdot append ( [ \begin{matrix}{x}_{{i}_{θ}}^{j} \\ {x}_{{i}_{γ}}^{j} \\ {x}_{i + {1}_{γ}}^{j} \\ o ( {r}_{i}^{j} ) \\ {x}_{i - {1}_{θ}}^{j} \\ {x}_{i - {1}_{γ}}^{j} \\ o ( {r}_{i}^{j} )\end{matrix} ] ) \\ μ ← data \cdot mean ( ) \\ Σ ← data \cdot covariance ( ) \\ \dot{μ} ← data \cdot mean ( ) \\ \dot{Σ} ← data \cdot covariance ( )$$

During operation, embodiments of the subject matter can execute the update model box above. The box describes two data stores, data and data both of which are initially set to empty (i.e. ø). These data stores can correspond to sets, lists, arrays, or any other structure capable of storing and retrieving data. The first loops handles the edge cases for each training sequence (n is the number of training examples). The second loop handles the interior (non-edge) cases for each training sequence (mⱼ is the number of amino acids in the sequence associated with training example j). In either case, the append operation adds to corresponding example to the training data. The superscripts here are used to denote a particular training example. For example, xᵢ_{_{θ}}^{j} corresponds to the i^{th} dihedral angle in an amino acid sequence from the j^{th} training example. Similarly, rᵢ^{j} corresponds to the i^{th} state value in an amino acid sequence from the j^{th} training example.

Subsequently, when all data has been appended, embodiments of the subject matter can determine the mean and covariance matrices of each set of training data. Multiple ways can be used to determine these matrices and to prevent singularity, a small value can be added along the diagonal of each covariance matrix.

Recall that the prediction model comprises two mean vectors and two covariance matrices. The undotted vectors and matrices correspond to the edge cases for training: the are based on data at the first and second amino acids, the state at the first amino acid, and the first dihedral angle. The dotted vectors and matrices correspond to the non-edge cases: they are based on data at all subsequence pairs of amino acids, states, and dihedral angles.

Embodiments of the subject matter can predict the most likely states for every pair of dihedral angles in a given sequence for each training example and then update the mean and covariance matrix based on those most likely states until convergence. These steps are shown in the box below. After embodiments of the subject matter execute the update model box, the next few steps are similar as in the prediction method in embodiments of the subject matter, except that the dihedral angles are known during learning.

Convergence can be defined in several ways. One way is with a fixed number of iterations of the above routine. Another way is until a difference of an aggregation of

$${max}_{s ∈ S} \{ {t}_{i , {m}_{j} , s} \}$$

over all training examples 1≤j≤n between successive iterations is less than a given threshold. Aggregation functions include but are not limited to mean, max, min, and sum. A difference can be absolute or relative. Convergence can also be defined as reaching a local maximum in likelihood.

$$\# repeat until convergence \\ update model \\ 1 \leq j \leq n : \\ s ∈ S : \\ {t}_{1 , s} ← ℒ ( [ \begin{matrix}{x}_{{1}_{θ}}^{j} \\ {x}_{{1}_{γ}}^{j} \\ {x}_{{2}_{γ}}^{j} \\ o ( s )\end{matrix} ] , μ , Σ ) \\ {g}_{1 , s} ← s \\ 2 \leq i \leq {m}_{j} - 1 : \\ s ∈ S : \\ {t}_{i , s} ← {max}_{{s}^{′} ∈ S} \{ l ( \begin{matrix}{x}_{{i}_{θ}}^{j} \\ {x}_{{i}_{γ}}^{j} \\ {x}_{i + {1}_{γ}}^{j} \\ o ( s )\end{matrix} \vert [ \begin{matrix}{x}_{i - {1}_{θ}}^{j} \\ {x}_{i - {1}_{γ}}^{j} \\ o ( {s}^{′} )\end{matrix} ] ) , θ : τ , θ : {τ}^{′} , \dot{μ} , \dot{Σ} ) + {t}_{i - 1 , {s}^{′}} \} \\ {g}_{i , s} ← {argmax}_{{s}^{′} ∈ S} \{ l ( \begin{matrix}{x}_{{i}_{θ}}^{j} \\ {x}_{{i}_{γ}}^{j} \\ {x}_{i + {1}_{γ}}^{j} \\ o ( s )\end{matrix} \vert [ \begin{matrix}{x}_{i - {1}_{θ}}^{j} \\ {x}_{i - {1}_{γ}}^{j} \\ o ( {s}^{′} )\end{matrix} ] ) , θ : τ , θ : {τ}^{′} , \dot{μ} , \dot{Σ} ) + {t}_{i - 1 , {s}^{′}} \} \\ {r}_{{m}_{j - 1}}^{j} ← {argmax}_{s ∈ S} \{ {t}_{{m}_{j} - 1 , s} \} \\ {m}_{j} - 1 \geq i \geq 2 : {r}_{i - 1}^{j} ← {g}_{{r}_{i}^{j}}$$

Multiple random restarts, each with different random state assignments can improve the probability of finding a global maximum in the likelihood. These multiple random restarts can be run in parallel and the model with largest aggregation of

$${max}_{s ∈ S} \{ {t}_{i , {m}_{j} , s} \}$$

over all training examples can be chosen as the best model. Alternatively, an ensemble of the top k models can be chosen.

Note that a mathematically equivalent version of the assignment for t and g can be defined in terms of a product of probabilities rather than a sum of log of the probabilities. The product of probabilities can result in extremely low numbers, which can cause hardware underflow. Hence in embodiments of the subject matter, the sum of the log of probabilities is preferred for reasons of greater precision. Moreover, with this form, the multivariate Gaussian distribution simplifies so that no exponentials are required. Other mathematically equivalent routines can be used as well as approximations of the multivariate Gaussian distribution.

An appropriate number of states (as in {1 . . . k}) can be determined in multiple different ways. For example, a validation set of sequences can be reserved and used to evaluate the likelihood of the sequences using an aggregation of

$${max}_{s ∈ S} \{ {t}_{i , {m}_{j} , s} \} .$$

The number of states can be explored from 1 . . . k until a maximum in the likelihood is found (the peak method) or until the likelihood does not significantly increase (the elbow method). These methods are similar to those of finding an appropriate number of mixtures for a Gaussian mixture distribution.

Embodiments of the subject matter spread local information globally in two ways. During prediction, both the state and the predicted dihedral angles at a location are used to predict both the state and the predicted dihedral angles at the subsequent location, the one immediately after. This propagation is guaranteed to be optimal even though the direction is from low to high location values. Hence, embodiments of the subject matter do not require repeated propagations as in deep learning. During learning, only the state information get propagated because the dihedral angles are known. In both cases, local information is spread globally, but more efficiently than in deep learning.

Embodiments of the subject matter can also be generalized to k^{th} order model between neighbors. For example, the formula to determine t_{i,s} can be based on the last two predecessors: s″, xᵢ₋₂_{_{γ}}, and a_{i-2,s∝}, where the maximization is over and s″ and t can include an extra state s′ as in

$${t}_{i , s , {s}^{′}} = {max}_{{s}^{′} ∈ S} \{ l ( [ \begin{matrix}{x}_{{i}_{γ}} \\ {x}_{i + {1}_{γ}} \\ o ( s )\end{matrix} ] \vert [ \begin{matrix}{a}_{i - 1 , {s}^{′}} \\ {x}_{i - {1}_{γ}} \\ o ( {s}^{′} ) \\ {a}_{i - 2 , {s}^{″}} \\ {x}_{i - {2}_{γ}} \\ o ( {s}^{″} )\end{matrix} ] , γ : τ , {θ}^{′} : {τ}^{″} ) + {t}_{i , {s}^{′} , {s}^{″}} \}$$

This example showed how embodiments of the subject matter can be extended to a second-order model. Extending to embodiments of the subject matter to a k^{th} order model involves adding additional successor states to t_{i,s,s′} as in t_{i,s,s′,s″,s″′ . . .} , adding successor data to the conditional part, including a larger selection of blocks in the conditional part, and shifting all the states over for the previously computed value of t. Theoretically, any higher-order model can be transformed into a first order model by adding more and more states.

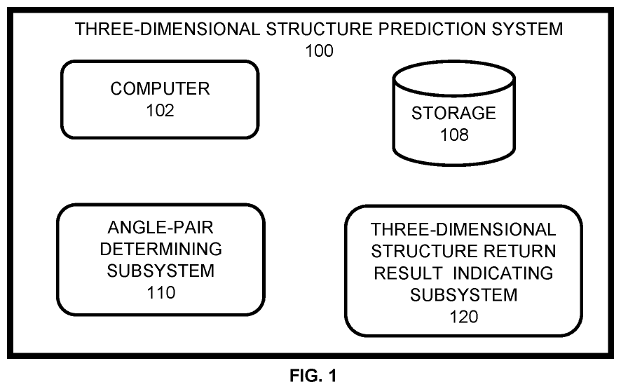

FIG. 1 shows an example three-dimensional structure prediction system 100 in accordance with an embodiment of the subject matter. Three-dimensional structure prediction system 100 is an example of a system implemented as a computer program on one or more computers in one or more locations (shown collectively as computer 102), with one or more storage devices (shown collectively as storage 108), in which the systems, components, and techniques described below can be implemented.

Three-dimensional structure prediction system 100 predicts the three-dimensional structure of an amino acid sequence. During operation, three-dimensional structure prediction system 100 determines, with angle-pair determining subsystem 110, a first pair of angles indexed by a first position in the sequence and a first state, based on a first amino acid indexed by the first position in the amino acid sequence, a second amino acid indexed by a second position the amino acid sequence, the first state, a second pair of angles indexed by a third position and a second state, a third amino acid indexed by a third position in the amino acid sequence, and the second state. Here, the first position is in proximity to the second position, and the first position is in proximity to the third position. Moreover, the second pair of angles indexed by the third position and the second state has previously been determined by dynamic programming

More specifically, angle-pair determining subsystem determines a_{i,s}, which corresponds to the first pair of angles, which are between xᵢ_{_{γ}} and xᵢ₊₁_{_{γ}}, which correspond to the first amino acid and the second amino acids, respectively. The sequence of amino acids corresponds to x. The first state corresponds to s. The second state corresponds to g_{i,s}, which has been determined with dynamic programming. The second pair of angles corresponds to a_{i-1,g}_{_{i,s}}, which are between xᵢ_{_{γ}} and xᵢ₋₁_{_{γ}}. Note that a_{i-1,g}_{_{i,s}}, which corresponds to the second pair of angles indexed by the third position and the second state, has previously been determined by dynamic programming. Also note that g_{i,s} has itself been previously determined by dynamic programming.

The locations for these angles and amino acids are referenced by i, i−1, and i+1. Here, i refers to the first position, i+1 refers to the second position, and i−1 refers to the third position. In this example, i (the first position) is in proximity to i+1 (the second position). Also in this example, i (the first position) is in proximity to i−1 (the third position).

Subsequently, three-dimensional structure prediction system 100 returns a result indicating the three-dimensional structure based on the first pair of angles with three-dimensional structure return result indicating subsystem 120. Clearly, any three-dimensional structure will at least include these first pair of angles in addition to all the other pairs of angles for the rest of the sequence.

The preceding description is presented to enable any person skilled in the art to make and use the subject matter, and is provided in the context of a particular application and its requirements. Various modifications to the disclosed embodiments will be readily apparent to those skilled in the art, and the general principles defined herein may be applied to other embodiments and applications without departing from the spirit and scope of the subject matter. Thus, the subject matter is not limited to the embodiments shown, but is to be accorded the widest scope consistent with the principles and features disclosed herein.

Embodiments of the subject matter and the functional operations described in this specification can be implemented in digital electronic circuitry, in tangibly embodied computer software or firmware, in computer hardware, including the structures disclosed in this specification and their structural equivalents, or in combinations of one or more of them.

Embodiments of the subject matter described in this specification can be implemented as one or more computer programs, i.e., one or more modules of computer program instructions encoded on a tangible non-transitory program carrier for execution by, or to control the operation of data processing system.

A computer program (which may also be referred to or described as a program, software, a software application, a module, a software module, a script, or code) can be written in any form of programming language, including compiled or interpreted languages, or declarative or procedural languages, and it can be deployed in any form, including as a stand-alone program or as a module, component, subroutine, or other unit suitable for use in a computing environment.

A computer program may, but need not, correspond to a file in a file system. A program can be stored in a portion of a file that holds other programs or data, e.g., one or more scripts stored in a markup language document, in a single file dedicated to the program in question, or in multiple coordinated files, e.g., files that store one or more modules, sub-programs, or portions of code.

Alternatively, or in addition, the program instructions can be encoded on an artificially generated propagated signal, e.g., a machine-generated electrical, optical, or electromagnetic signal, that is generated to encode information for transmission to a suitable receiver system for execution by a data processing system. The computer storage medium can be a machine-readable storage device, a machine-readable storage substrate, a random or serial access memory device, or a combination of one or more of them.

Computers suitable for the execution of a computer program include, by way of example, can be based on general or special purpose microprocessors or both, or any other kind of central processing unit. Generally, a central processing unit will receive instructions and data from a read only memory or a random-access memory or both. The essential elements of a computer are a central processing unit for performing or executing instructions and one or more memory devices for storing instructions and data.

A computer can also be distributed across multiple sites and interconnected by a communication network, executing one or more computer programs to perform functions by operating on input data and generating output.

A computer can also be embedded in another device, e.g., a mobile telephone, a personal digital assistant (PDA), a mobile audio or video player, a game console, a Global Positioning System (GPS) receiver, or a portable storage device, e.g., a universal serial bus (USB) flash drive, to name just a few.

Generally, a computer will also include, or be operatively coupled to receive data from or transfer data to, or both, one or more mass storage devices for storing data, e.g., magnetic, magneto optical disks, or optical disks. However, a computer need not have such devices.

The term “data processing system’ encompasses all apparatus, devices, and machines for processing data, including by way of example a programmable processor, a computer, or multiple processors or computers.

For a system of one or more computers to be configured to perform particular operations or actions means that the system has installed on it in software, firmware, hardware, or a combination of them that in operation cause the system to perform the operations or actions. For one or more computer programs to be configured to perform particular operations or actions means that the one or more programs include instructions that, when executed by data processing system, cause the system to perform the operations or actions.

The processor and the memory can be supplemented by, or incorporated in, special purpose logic circuitry. More generally, the processes and logic flows can also be performed by and be implemented as special purpose logic circuitry, e.g., an FPGA (field programmable gate array) or an ASIC (application specific integrated circuit), a dedicated or shared processor that executes a particular software module or a piece of code at a particular time, and/or other programmable-logic devices now known or later developed. When the hardware modules or system are activated, they perform the methods and processes included within them.

The system can also include, in addition to hardware, code that creates an execution environment for the computer program in question, e.g., code that constitutes processor firmware, a protocol stack, a database management system, an operating system, or a combination of one or more of them.

The computer-readable storage medium includes, but is not limited to, volatile memory, non-volatile memory, magnetic and optical storage devices such as disk drives, magnetic tape, CDs (compact discs), DVDs (digital versatile discs or digital video discs), computer instruction signals embodied in a transmission medium (with or without a carrier wave upon which the signals are modulated), and other media capable of storing computer-readable media now known or later developed. For example, the transmission medium may include a communications network, such as a LAN, a WAN, or the Internet.

Computer readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD-ROM disks.

The methods and processes described in the detailed description section can be embodied as code and/or data, which can be stored in a computer-readable storage medium as described above. When a computer system reads and executes the code and/or data stored on the computer-readable storage medium 120, the computer system performs the methods and processes embodied as data structures and code and stored within the computer-readable storage medium.

The components of the system can be interconnected by any form or medium of digital data communication, e.g., a communication network. Examples of communication networks include a local area network (“LAN”) and a wide area network (“WAN”), e.g., the Internet.

While this specification contains many specific implementation details, these should not be construed as limitations on the scope of any subject matter or of what may be claimed, but rather as descriptions of features that may be specific to particular embodiments of particular subject matters. Certain features that are described in this specification in the context of separate embodiments can also be implemented in combination in a single embodiment.

Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or variation of a sub-combination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous.

Moreover, the separation of various system modules and components in the embodiments described above should not be understood as requiring such separation in all embodiments, and it should be understood that the described program components and systems can generally be integrated together in a single software product or packaged into multiple software products.

The foregoing descriptions of embodiments of the subject matter have been presented only for purposes of illustration and description. They are not intended to be exhaustive or to limit the subject matter to the forms disclosed. Accordingly, many modifications and variations will be apparent to practitioners skilled in the art. Additionally, the above disclosure is not intended to limit the subject matter. The scope of the subject matter is defined by the appended claims.

What is claimed is:

1. A computer-implemented method for facilitating predicting a three-dimensional structure of an amino acid sequence comprising:
determining a first pair of angles indexed by a first position in the sequence and a first state, based on a first amino acid indexed by the first position in the amino acid sequence, a second amino acid indexed by a second position the amino acid sequence, the first state, a second pair of angles indexed by a third position and a second state, a third amino acid indexed by a third position in the amino acid sequence, and the second state,
wherein the first position is in proximity to the second position,
wherein the first position is in proximity to the third position, and
wherein the second pair of angles indexed by the third position and the second state has previously been determined by dynamic programming; and
returning a result indicating the three-dimensional structure of the amino acid sequence based on the first pair of angles.

2. The method of claim 1,
wherein determining the first pair of angles is based on a multivariate Gaussian distribution comprising a mean vector and a covariance matrix.

3. The method of claim 2,
wherein the mean vector and the covariance matrix are learned from training data comprising at least a third amino acid, a fourth amino acid, a third state associated with the third amino acid, and a third pair of angles associated with the third amino acid.

4. The method of claim 3,
wherein the mean vector and the covariance matrix are learned from training data comprising one-hot representations of the third state and the third and fourth amino acids.

5. One or more non-transitory computer-readable storage media storing instructions that when executed by one or more computers cause the one or more computers to perform operations for facilitating predicting a three-dimensional structure of an amino acid sequence, comprising:
determining a first pair of angles indexed by a first position in the sequence and a first state, based on a first amino acid indexed by the first position in the amino acid sequence, a second amino acid indexed by a second position the amino acid sequence, the first state, a second pair of angles indexed by a third position and a second state, a third amino acid indexed by a third position in the amino acid sequence, and the second state,
wherein the first position is in proximity to the second position,
wherein the first position is in proximity to the third position, and
wherein the second pair of angles indexed by the third position and the second state has previously been determined by dynamic programming; and
returning a result indicating the three-dimensional structure of the amino acid sequence based on the first pair of angles.

6. The one or more non-transitory computer-readable storage media of claim 5,
wherein determining the first pair of angles is based on a multivariate Gaussian distribution comprising a mean vector and a covariance matrix.

7. The one or more non-transitory computer-readable storage media of claim 6,
wherein the mean vector and the covariance matrix are learned from training data comprising at least a third amino acid, a fourth amino acid, a third state associated with the third amino acid, and a third pair of angles associated with the third amino acid.

8. The one or more non-transitory computer-readable storage media of claim 7,
wherein the mean vector and the covariance matrix are learned from training data comprising one-hot representations of the third state and the third and fourth amino acids.

9. A system comprising one or more computers and one or more storage devices storing instructions that when executed by the one or more computers cause the one or more computers to perform operations for facilitating predicting a three-dimensional structure of an amino acid sequence, comprising:
determining a first pair of angles indexed by a first position in the sequence and a first state, based on a first amino acid indexed by the first position in the amino acid sequence, a second amino acid indexed by a second position the amino acid sequence, the first state, a second pair of angles indexed by a third position and a second state, a third amino acid indexed by a third position in the amino acid sequence, and the second state,
wherein the first pair of angles is indexed by the first state and a first location,
wherein the first position is in proximity to the second position,
wherein the first position is in proximity to the third position, and
wherein the second pair of angles indexed by the third position and the second state has previously been determined by dynamic programming; and
returning a result indicating the three-dimensional structure of the amino acid sequence based on the first pair of angles.

10. The system of claim 9,
wherein determining the first pair of angles is based on a multivariate Gaussian distribution comprising a mean vector and a covariance matrix.

11. The system of claim 10,
wherein the mean vector and the covariance matrix are learned from training data comprising at least a third amino acid, a fourth amino acid, a third state associated with the third amino acid, and a third pair of angles associated with the third amino acid.

12. The system of claim 11,
wherein the mean vector and the covariance matrix are learned from training data comprising one-hot representations of the third state and the third and fourth amino acids.
